(19) United States
(12) Patent Application Publication
(10) Pub. No.: US 2025/0099190 A1
(43) Pub. Date: Mar. 27, 2025

(54) MEDICAL INSTRUMENT ARRANGEMENT FOR THE MINIMALLY INVASIVE EXAMINATION AND/OR TREATMENT OF A SUBJECT, METHOD FOR OPERATING A MEDICAL INSTRUMENT ARRANGEMENT, COMPUTER PROGRAM, AND ELECTRONICALLY READABLE DATA MEDIUM

(71) Applicant: Siemens Healthineers AG, Forchheim (DE)

(72) Inventor: Marcus Pfister, Bubenreuth (DE)

(21) Appl. No.: 18/893,607

(22) Filed: Sep. 23, 2024

(30) Foreign Application Priority Data
Sep. 26, 2023 (DE) 10 2023 209 397.9

(51) Int. Cl. A61B 34/32; G05B 13/02

(52) U.S. Cl. CPC A61B 34/32; G05B 13/0265

(57) ABSTRACT

A medical instrument arrangement includes: a medical instrument having a proximal operational section for moving the instrument; a robot for active manipulation of the medical instrument on the operational section; an operational capture device for capturing operator movement patterns of the operational section of the medical instrument generated manually; and a control device. The control device is configured to: control the robot in accordance with control commands for generating corresponding movements of the operational section; determine an intervention situation for captured operator movement patterns; store captured operator movement patterns with the assigned intervention situation; and automatically select and/or generate a control movement pattern from stored operator movement patterns and/or from evaluation movement patterns deduced therefrom as a function of a present intervention situation of the situation capture unit and for the automatic implementation of the control movement pattern as control commands using the control unit.

The present patent document claims the benefit of German Patent Application No. 10 2023 209 397.9, filed Sep. 26, 2023, which is hereby incorporated by reference in its entirety.

### TECHNICAL FIELD

The disclosure relates to a medical instrument arrangement for the minimally invasive examination and/or treatment of a subject, wherein the medical instrument arrangement includes a medical instrument configured to be introduced into the subject, the medical instrument having a proximal operational section for moving the instrument. The medical instrument arrangement further includes a robot for the active manipulation of the medical instrument on the operational section. The disclosure additionally relates to a method for operating such an instrument arrangement, a computer program, and an electronically readable data medium.

### BACKGROUND

During the minimally invasive examination and/or treatment of a subject, in particular a patient, (referred to as a minimally invasive intervention or intervention for short), a medical instrument, (e.g., a catheter, a guide wire, or the like), is introduced into the subject, (e.g., into a vascular system). The medical instrument may i have at least one sensor for the examination and/or at least one treatment agent for the treatment.

One example of such minimally invasive interventions is treatments and/or examinations in the vascular system of a patient with a catheter as a medical instrument, (e.g., for dissolving total vascular occlusions (CTO), for introducing stents, and the like). The catheter or the medical instrument, (e.g., including a guide wire), may be introduced via the patient's groin, from where it is guided to the actual site of the intervention, i.e., the site of the examination and/or treatment. This has hitherto mostly been done manually, by rotating, translating (feed/retraction), or curving the medical instrument proximally outside the subject on an operational section. For navigation, besides pre-interventional image datasets and position determination systems for the instrument, fluoroscopy images or other intra-interventional images are mostly acquired, which may then be superimposed on a pre-interventional three-dimensional image dataset. While the navigation to the site of the intervention may itself prove difficult, there are particular maneuvers that may aid the navigation, examination, and/or treatment and which depend in particular on the experience of the operator, i.e., the person performing the intervention. This then results in complex movement patterns.

Examples of cases in which complex movement patterns for the operation of the medical instrument may occur include navigation into small vascular branches far away from the entry point of the medical instrument into the subject, (e.g., in neuroradiological procedures in the brain), and intricate navigation when opening up total occlusions.

To simplify the performance of minimally invasive interventions, it has been proposed to use robots, which take over the management of the medical instrument on the operational section from the operator and actively manipulate the medical instrument. The robot in turn is controlled by the operator via corresponding operating mechanisms or devices, (e.g., a joystick), in order to generate rotational and/or translatory movements or curvatures of the medical instrument, wherein although every medical instrument may be rotated and translated, only correspondingly designed medical instruments may be curved. Such operating mechanisms or devices make it more difficult to continue to apply movement patterns for the medical instrument that have been learned over a long period of time and/or trained by experience. Although automated movement patterns for robots have already been proposed for the movement of medical instruments, this merely relates to a few simple movement patterns such as predefined translations or rotations on a small scale.

In summary, medical instruments, (e.g., catheters), may currently either be controlled directly manually or indirectly via robots, wherein in the latter case it is only possible to use a few standard movements.

### SUMMARY AND DESCRIPTION

The object of the disclosure is to specify an improved, more far-reaching and in particular individualizable usability of robots in minimally invasive interventions with medical instruments.

This object is achieved by a medical instrument arrangement, a method, a computer program, and an electronically readable data medium as disclosed herein. The scope of the present disclosure is defined solely by the appended claims and is not affected to any degree by the statements within this summary. The present embodiments may obviate one or more of the drawbacks or limitations in the related art.

In accordance with the disclosure, a medical instrument arrangement for the minimally invasive examination and/or treatment of a subject includes: a robot for the active manipulation of a medical instrument that may be introduced into the subject and has a proximal operational section for active manipulation, on the operational section; an operational capture device for capturing operator movement patterns of the operational section of the medical instrument generated manually, in particular directly on the operational section; and a control device. The control device includes: a control unit for controlling the robot in accordance with control commands for generating control movements of the operational section; a situation capture unit for determining an intervention situation to be assigned to captured operator movement patterns; a memory for the, in particular operator-related, storage of captured operator movement patterns with the assigned intervention situation in a storage device of the control device; and a situation unit for automatically selecting and/or generating a control movement pattern from stored operator movement patterns as a function of an intervention situation determined by the situation capture unit and for providing the control movement pattern as control commands to control the robot using the control unit.

In this case, the subject may be a human and/or animal patient, as well as a phantom, in particular a practice phantom. In particular, at different points in time a different subject may be used, for example, a practice phantom in a first application phase of the medical instrument arrangement, namely a training phase. In particular, it may therefore be provided that the instrument arrangement further also includes a phantom as a subject, on which at least some of the operator movement patterns are captured.

The medical instrument may be a catheter and/or a guide wire, or another medical instrument, in order to perform a minimally invasive intervention, e.g., an examination and/or treatment using the medical instrument.

The operational capture device, which may be a recorder device, is used in the aforementioned first application phase, namely the training phase, to capture the manual movement by the operator, in particular taking place directly on the operational section. The operational section of the medical instrument need not be a permanently defined, immovable section of the instrument, but may change depending on the feed distance into the subject, for example, if the instrument may be gripped directly or by a movable gripper sleeve or the like. In particular, the operational section is consequently a part of the medical instrument that projects out of the subject and is in principle present, at the entry point, consequently proximally, for minimally invasive interventions.

The operational capture device supplies capture data that describes the operator movement patterns realized by the operator. Since rotations and translations (feed/retraction) and/or curvatures of the medical instrument may be carried out on the operational section of the medical instrument, in particular by the operator's fingers, an expedient development of the present disclosure provides that the capture data includes a course of rotation and course of translation and/or course of curvature brought about by the operating actions of the operator. The control commands may include a course of rotation and course of translation and/or course of curvature for the instrument, in particular its operational section, to be implemented by the control unit.

In accordance with the disclosure, it is therefore proposed to transfer movement patterns from at least one operator during the manual guidance of the medical instrument on the operational section to the control of the robot, such that these may be provided automatically on a situation-specific basis. In other words, the robot movements are adjusted to the movement patterns of the medical personnel for a particular intervention situation. The medical instrument arrangement enables, in a self-learning manner, special specific movement patterns of an operator to be integrated into a robotic minimally invasive intervention and thus the movement repertoire of the robot to be extended. Special movements by the at least one operator are learned together with the intervention situation and may be retrieved automatically in a corresponding intervention situation by the robot, more specifically the control device.

In a training phase, during manual operation of the medical instrument by an operator, in particular on the operational section itself, it is made possible to learn operator movement patterns by recording using the operational capture device and to store them using the memory of the control device. Here, not only are the operator movement patterns, for example, a sequence of translations and rotations and/or curvatures, learned, but with these an intervention situation, in which the operator movement pattern is used, is captured, assigned to the operator movement pattern, and stored. The training phase expediently takes place without any use of the robot, instead using the operational capture device (recorder device), which may be arranged at the instrument airlock to the subject.

The operational capture device may here specifically be configured in different ways. For example, it may have drivers, for example rollers, whose movement is measured, and which are moved by the operational section of the medical instrument by friction or other interaction. In an embodiment, markers are provided on the operational section of the instrument, which may be captured via a corresponding optical, sensor system of the operational capture device.

In a second application phase of the medical instrument arrangement, which may be referred to as the recall phase, the presence of a particular intervention situation is automatically identified. A corresponding control movement pattern, which may be an operator movement pattern, or else may be derived therefrom, is retrieved and executed, in particular after confirmation by an operator.

In this case, the medical instrument arrangement may work operator-specifically, to which end the operator is expediently stored with the operator movement pattern and the assigned intervention situation. In particular, the situation unit is configured for the operator-related selection and/or generation of the control movement pattern stored on the basis of just the operator movement patterns relating to the current operator. In this way, the individual movement patterns of particular operators are learned, which the operator particularly trusts due to his or her own authorship and ultimately recognizes in the robot's mode of operation. The specific experience and approach of the operator is then thus reflected in the automatic work of the robot in accordance with the control movement pattern.

In this case, the learning/recording of particular operator movement patterns takes place differently. In particular, when using a phantom, explicit, targeted learning may also take place using the medical instrument arrangement. To this end, the operator may manually start a movement recording, for example, activate the operational capture device in a special learning mode via the control device and intentionally perform the corresponding movements of the operator movement pattern to be recorded, in particular on the phantom as subject. In this connection, the intervention situation may likewise be provided by the operator, for example, via a user interaction unit of the control device.

In certain embodiments, minimally invasive interventions of the operator are captured by the medical instrument arrangement, in particular during practice on the phantom and/or actual minimally invasive interventions on a patient. In this connection too, different approaches are conceivable.

Thus, in a first concrete approach of learning by observing the operator, the operational capture device is configured for the continuous capture of the operational activity during an intervention on the subject and the situation capture unit is configured for subsequent situation assignment and corresponding selection of operator movement patterns from the operational activity. This means that the movements the operator performs may be captured continuously, stored in the storage device and in a subsequent analysis assigned at least in part to an intervention situation. In this analysis, the operator may “review” the intervention and his or her performance, wherein, in particular with simultaneous storage of additional information, an automatic analysis of such a recording of the operator activity during a minimally invasive intervention may also take place.

In a second concrete approach of learning by observing, the control device has an activation unit for activating the operational capture device and/or for activating the storage of operator movement patterns on fulfillment of an activation condition, which evaluates a current intervention situation and/or a current operating pattern and/or instrument movement pattern of the instrument captured by the situation capture unit. This means that the recording may be started automatically by the medical instrument arrangement if a corresponding activation condition is present, for example, if it has been established that a particular intervention situation has occurred for which at least one operator movement pattern is to be acquired. Additionally, or alternatively, it is also conceivable to specifically detect “unusual” behavior, in particular operating patterns or movement patterns that deviate from the normal feed to the site of the intervention, and to store them correspondingly. In this case, the operating and/or instrument movement pattern relates in particular to an operating period which ends at the current point in time.

In particular, if merely the (final) storage is to be controlled, (e.g., the operational capture device is in principle active during the minimally invasive intervention), it may be expedient to use a circulating memory, in which the most recently captured capture data of the operational capture device may be stored, in order for example to be evaluated by the activation condition and at least partially also to be taken over into the overall dataset (containing at least the operator movement patterns and the intervention situation) to be finally stored in the storage device.

However, variants are also conceivable in which the operational capture device need not be continuously operated, in particular if another capture of the behavior of the operator is enabled and/or at least the present intervention situation is continuously monitored. For example, an unusual movement may also be captured by imaging and the like, in particular if imaging monitoring of the minimally invasive intervention, for example by fluoroscopy, takes place. It is further also conceivable to employ additional sensors, (e.g., cameras), which monitor the minimally invasive intervention. In particular, the analysis of the operating and/or instrument movement pattern is used, as mentioned, for the detection of unusual behavior deviating from operating and/or movement patterns for the normal feed and if appropriate retraction. In respect of the intervention situation, the activation condition may monitor whether an intervention situation from a predefined list of intervention situations leading to activation is present, in particular the initial state thereof.

In certain embodiments, the activation unit is configured, for the evaluation of the activation condition, to compare the current operating and/or instrument movement pattern with at least one predefined normal pattern, which in particular describes a normal feed operation of the instrument, wherein the activation condition is fulfilled if a deviation exceeds a threshold value for each normal pattern. For example, correlation values of the current operating and/or instrument movement pattern with the normal patterns may be determined, the activation condition being fulfilled if a threshold value for the correlation value for all normal patterns is exceeded. It is further also conceivable to employ an, in particular trained, pattern classification function, which classifies an operating and/or instrument movement pattern as “normal” or “unusual.” It may be provided that the normal patterns are deduced from training data. In other words, using training data “normal” movements of the operator may be learned and recording may be started if the current movements deviate from this.

It may be noted at this point that, in respect of the intervention situation too, an, in particular trained, classifier may be employed by the situation capture unit, which is discussed in greater detail below.

In certain examples, the instrument arrangement may have at least one sensor for capturing sensor data for determination of the current operating and/or instrument movement pattern by the activation unit and/or for determination of the present intervention situation by the situation capture unit. For example, a camera is used as a sensor, which is directed at the operator's hands. However, other comparable sensor data may also be employed. For example, the instrument movement pattern may also relate to the tip of the medical instrument, as is visible for example in image data.

Finally, in certain cases, the situation capture unit may be configured to determine the intervention situation at least partially using an operator input. This may be expedient when introducing new intervention situations and/or when it is not otherwise possible to determine the intervention situation. For example, a user may define a new intervention situation or intervention situation class and start the recording for this as it were manually. This may be particularly expedient for the above-mentioned “dry run” on a phantom or the like.

Different approaches are also conceivable for the assignment of intervention situations to operator movement patterns. As already mentioned, explicit learning by the operator is initially possible, wherein the operator assigns the operator movement pattern explicitly to an intervention situation.

It is further possible to observe procedures of the operator, wherein, on activation using the activation unit information describing the intervention situation, for example a position of the instrument and/or anatomical information, in particular a position of the instrument, in particular the tip of the instrument, relative to the surrounding anatomy, may be already assigned to the operator movement pattern to be acquired. A different anatomical assignment is also possible, for example, by determining (if appropriate also subsequently, with additional logged information assigned to the operator movement pattern) the anatomical background against which the medical instrument is located and/or whether a particular landmark, for example in an overlay, is reached.

In certain examples, the capture unit is configured to capture the intervention situation for an operator movement pattern for the definition of a situation dataset, which describes at least the instrument position relative to the surrounding anatomy, in particular in respect of at least one landmark, and to determine the present intervention situation by comparison with stored situation datasets. Overall, it is expedient if the situation capture unit is configured to use such situation datasets, which define the intervention situation and at least describe the instrument position relative to the surrounding anatomy, in particular the position of the tip of the instrument relative to the surrounding anatomy. In particular, new intervention situations may arise if the intervention situation is not yet known, consequently if no (sufficient) match is found during the comparison. Such intervention situations may include that the tip of the medical instrument is located against an anatomical feature which requires more complex movement patterns, for example against a bifurcation, a vascular branch, or a total occlusion.

Accordingly, intervention situations and assigned movement patterns may also include the opening up of a total occlusion (CTO), navigation into a vascular branch, in particular exploration of a bifurcation and/or branching mapped only two-dimensionally, and navigation of small vessels (“fingertip technique”).

In respect of the situation dataset, an expedient development provides that the situation capture unit is configured to store the situation dataset with the assigned operator movement pattern and/or the situation dataset includes image data showing the instrument in the anatomy and/or position data from a position capture device for the medical instrument. In this case, the landmark may be highlighted in the image data with a landmark reference. The storage of the situation dataset, in particular in connection with the image data and/or position data, is in particular also useful if the intervention situation is to be automatically determined in the second application phase (recall phase), in order to select and/or generate a corresponding control movement pattern. It may then be provided that the medical instrument and/or the anatomical region and/or the attainment of the landmark is identified in current image data, in order thus to be able to deduce the corresponding intervention situation in comparison with stored intervention situations or specifically the situation datasets. The control movement pattern may then be selected and in particular suggested.

It may again be noted at this point that the position data of the medical instrument may relate to the tip of the instrument, as far as the intervention situation is concerned, but it is also additionally or alternatively conceivable for position data of the medical instrument to include system positions, for example a current feed into the subject.

Additionally, or alternatively to the use of situation datasets and/or an image analysis for determining an intervention situation or implementing these variants, the situation capture unit may be configured to use at least one, in particular trained, classification function for identifying the intervention situation. The input data for such a trained classification function may include capture data from the operator capture device (for example capture data stored in a circulating memory). However, it also expediently includes at least one piece of additional information, in particular position data from a position capture device for the medical instrument and/or the robot and/or image data from an imaging device and/or settings data of components of the instrument arrangement. In particular, the above situation dataset may also be used to classify a current intervention situation. Correspondingly, the output data of such a classification function may include the classification into an intervention situation class. In this connection too, it may be expedient for at least one intervention situation and/or intervention situation class to be defined via an instrument position relative to a surrounding anatomy.

In certain examples, a trained function maps cognitive functions that human beings associate with other human brains. Using training based on training data (machine learning), the trained function is able to adjust to new circumstances and to detect and extrapolate patterns.

In certain examples, parameters of a trained function may be adjusted by training. In particular, supervised learning, semi-supervised learning, unsupervised learning, reinforcement learning and/or active learning may be used. In addition, representation learning (also known as “feature learning”) may be employed. The parameters of the trained function may be adjusted iteratively using multiple training acts.

A trained function may include a neural network, a support vector machine (SVM), a decision tree, and/or a Bayes network. In certain examples, the trained function may be based on k-means clustering, Q-learning, genetic algorithms, and/or assignment rules. In particular, a neural network may be a deep neural network, a convolutional neural network (CNN) or a deep CNN. In addition, the neural network may be an adversarial network, a deep adversarial network and/or a generative adversarial network (GAN).

It is therefore also possible that, in the second application phase (recall phase), an intervention situation may be identified using current movement patterns, as they are brought about by the operator using the robot. Consequently, the control device, in particular the situation capture unit, may identify whether the operator is trying to execute one of his or her special movement patterns, for example, a “special sequence,” with the robot. The current intervention situation may be deduced from this, in order then to suggest the control movement pattern.

For the concrete implementation of suitable automatisms on identification of an intervention situation for which a control movement pattern may be selected and/or generated and if appropriate suggested, an expedient development of the present disclosure provides that the control device further has a trigger unit that triggers the selection and/or generation of the situation unit on fulfillment of a trigger condition evaluating the present intervention situation of the situation detection unit. In particular, the trigger condition may check whether operator movement patterns and/or evaluation movement patterns, to which a corresponding intervention situation is assigned, are already stored in the storage device for the current intervention situation. In summary, it is therefore automatically monitored whether an intervention situation is occurring for which a control movement pattern may be provided, whereby this provision is achieved automatically due to the trigger unit.

The control device may expediently have a user interaction unit to suggest the control movement pattern to the operator, in particular when triggering, and/or for the receipt of an operator input, in particular for use in the selection. A suggestion function, which due to the current intervention situation automatically opens up further, in particular individualized, operating options for the robot, is in particular advantageous here and also brings about a confirmation by the operator.

In certain examples, the control device has an evaluation unit for the statistical evaluation of multiple operator movement patterns for a similar intervention situation present in the storage device for determining a selectable evaluation movement pattern for this intervention situation. Consequently, if multiple operator movement patterns, in particular of the same operator, are present for an intervention situation, a statistical evaluation may take place, in order to derive the essence of the multiple captures, in particular thus the actually desired movement properties, and in particular to deduce an optimal evaluation movement pattern, which may then be selected or used by the situation unit for the intervention situation (and if appropriate the operator). In particular, online generation, for example, directly prior to the selection and/or generation in the situation unit, is also conceivable here. This in particular enables a determination of improved and also updateable control movement patterns, because it is possible to learn from multiple processes relating to the same intervention situation.

The statistical evaluation may, in respect of similar sections of the movement pattern, include messages in respect of the duration and/or amplitude and/or course of the movement, in particular for translation and rotation and/or curvatures. However, it is also possible to determine partial movement patterns statistically for the intervention situation and to base the evaluation movement pattern thereon.

In this connection, it is in particular advantageous if the memory is further configured for the, in particular time-offset, storage of success information describing the success of the respective operator movement pattern, in particular as a continuous efficiency variable, for each operator movement pattern, wherein the evaluation unit is configured to determine the evaluation movement pattern as an optimal movement pattern using the success information. In this embodiment, the efficiency and/or success of the treatment, described by the success information, are taken into account, wherein an evaluation movement pattern optimized for the respective intervention situation comes about based on the success information. The success information makes it possible to identify the most efficient parts of the operator movement patterns in the statistical evaluation, for example, partial movement patterns present in particularly successful interventions or pronounced partial movement patterns, and to be made the subject of the evaluation movement pattern. In averaging procedures in connection with the statistical evaluation, a weighting may be carried out as a function of the success information. In addition, operator movement patterns resulting in a low level of success may also be excluded. In this case, the success information may be continuous or at least graduated success information, (e.g., an efficiency variable), and thus not binary success information. For example, a percentage efficiency or another score may be determined and used as success information, in particular an efficiency variable.

In this connection, an expedient development of the medical instrument arrangement provides that the evaluation unit is further also configured to evaluate image data of the instrument and of the surrounding anatomy recorded during and/or after the performance of the operator movement pattern for the at least partial automatic determination of the success information. In other words, it is conceivable for the success information to be deduced automatically by analysis from observation results for the intervention in which the operator movement pattern was employed. Here, an image-based evaluation is in particular an option. If, for example, there is an intervention in the vascular system of a patient as an application, it is possible to assess from image data, for example, whether a vessel has been punctured and/or to what extent a total occlusion has been removed. However, it is also conceivable to include user inputs and/or other information in such a postoperative evaluation.

Evaluation movement patterns determined in a previous evaluation and their success information may expediently also be used in the evaluation. This is in particular expedient in updatings. Using success information for evaluation movement patterns used in particular as control movement patterns, it is also possible to assess how good the statistical evaluation was and if appropriate may be improved.

The control device may further have an adjustment unit configured to adjust the operator movement patterns and/or evaluation movement patterns to be stored in respect of at least one restriction of movement relating to the robot and/or the protection of the subject. In this way, it is also possible to include stipulations for the protection of the subject and/or specifications of the robot in the learning procedure, so that the learned operator movement patterns and/or evaluation movement patterns may also actually be executed by the robot. The control device may be “smart” and may take account of restrictions regarding the robot and/or the subject. For example, if it is known that the robot cannot exceed a limit value for the number of revolutions per second during the rotation, a value of the operator movement pattern and/or evaluation movement pattern exceeding the limit value may be brought down to the limit value. The same also applies in respect of the feed/retraction.

Besides the medical instrument arrangement, a computer-implemented method is provided for operating a medical instrument arrangement for the minimally invasive examination and/or treatment of a subject, having a medical instrument that may be introduced into the subject and a proximal operational section for moving the instrument. The method includes: capturing operator movement patterns of the operational section of the medical instrument that are generated manually, in particular directly on the operational section; assigning an intervention situation to each captured operator movement pattern, storing (e.g., operator-related storing) the captured operator movement patterns with the assigned intervention situation; and, in a further intervention with the medical instrument and a robot for the active manipulation of the medical instrument on the operational section, automatically selecting and/or generating a control movement pattern from stored operator movement patterns as a function of an intervention situation present; and automatically implementing the control movement pattern as control commands for the robot.

All explanations regarding the medical instrument arrangement may be transferred analogously to the method and vice versa, so that here too the aforementioned advantages may be retained.

A computer program may be loaded directly into a storage device of a control device of a medical instrument arrangement and has program code or means such that, on execution of the computer program in the control device, the control device is caused to execute the acts of a method described herein. The computer program may be stored on an electronically readable data medium, which consequently includes control information stored thereon, which includes a computer program and is embodied such that when the data medium is used in a control device of an instrument arrangement this is configured to execute a method described herein. The data medium is in particular a non-transient data medium, for example a CD-ROM.

### BRIEF DESCRIPTION OF THE DRAWINGS

Further advantages and details of the present disclosure emerge from the exemplary embodiments described below, and on the basis of the drawings, in which:

FIG. 1 depicts a schematic sketch of an example of a medical instrument arrangement.

FIG. 2 depicts the functional structure of an example of a control device of the instrument arrangement in FIG. 1.

FIG. 3 depicts a flow chart of an exemplary embodiment of an operating procedure.

FIG. 4 depicts an illustration of an example of an intervention situation.

FIG. 5 depicts an exemplary operator movement pattern.

FIG. 6 schematically depicts an example of the implementation of the operator movement pattern as a control movement pattern for a robot.

### DETAILED DESCRIPTION

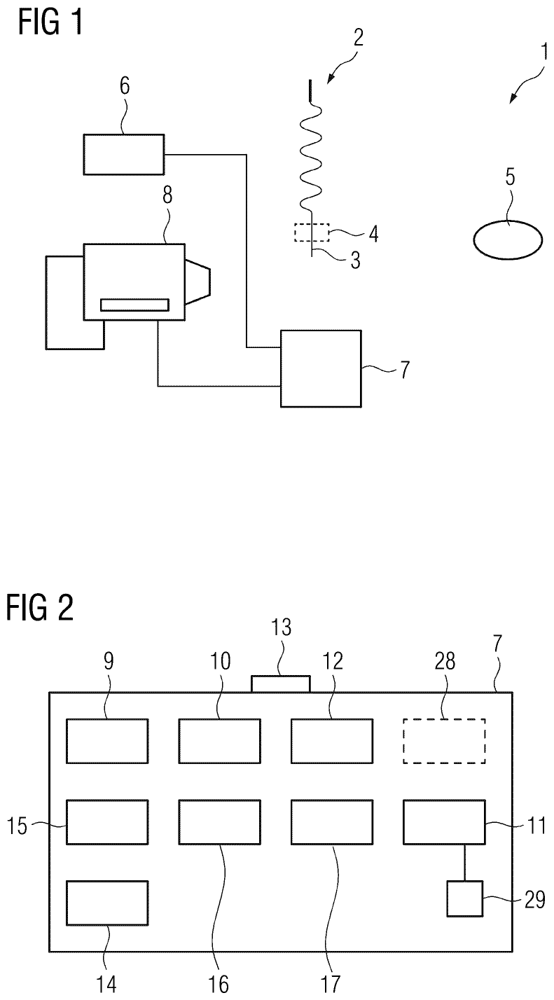

FIG. 1 shows a schematic sketch of a medical instrument arrangement 1 for the performance of minimally invasive interventions with a medical instrument 2, (e.g., a catheter or a guide wire). The medical instrument 2 has a proximal operational section 3, in particular the section of the instrument 2 in each case located just outside a subject. The instrument 2 may be introduced into a subject 5, (e.g., a patient or a phantom), via an instrument airlock 4 at an entry point.

The medical instrument 2 may be operated on the operational section 3 directly by an operator, for example, for feed/retraction (translation) and in respect of the rotation and/or curvature of the medical instrument 2. To be able to capture such operator movement patterns brought about by an operator, the medical instrument arrangement 1 further has an operational capture device 6 that has corresponding measurement mechanisms or devices, for example, in order to capture markers on the instrument 2 or otherwise track the movement on the operational section 3. During manual guidance of the instrument 2 on the operational section 3 by an operator, capture data describing the operator movement pattern may be captured and passed to a control device 7. To be able to record operator movement patterns selectively, the phantom 5 may be used. It is also possible to record operator movement patterns during minimally invasive interventions on other subjects.

The medical instrument arrangement 1 also has a robot 8, (e.g., a vascular robot), which may be coupled to the operational section 3 in order to bring about movements, in accordance with control commands that may be based on an operator input at an operating mechanism or device or else a different source, of the medical instrument 2, in this case again rotations and translations and/or curvatures.

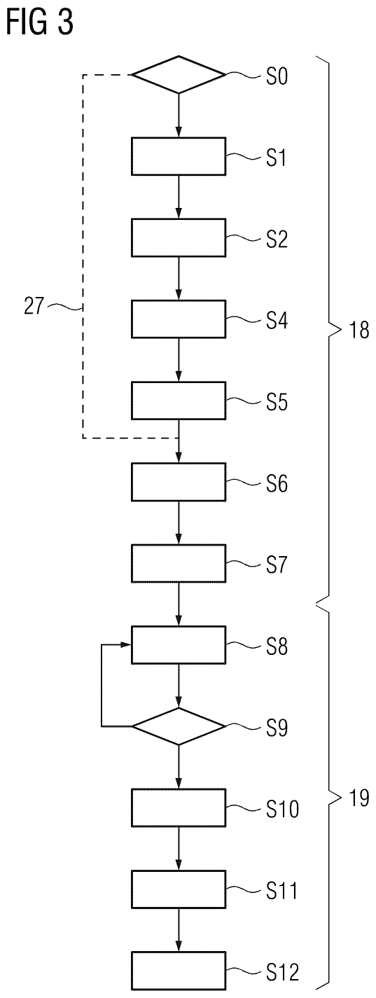

FIG. 2 shows the functional structure of the control device 7 in greater detail. In this case, the various functional units of the control device 7 are first presented in FIG. 2, before their coherent operation is explained in greater detail with the help of the method in FIG. 3.

The control device 7 first has a control unit 9 configured to control the robot 8 in accordance with control commands for the generation of corresponding movements, here translations and rotations and/or curvatures.

In the present embodiment, the control device 7 further has an activation unit 10 that controls the activity of various other functional units, but in particular also the operational capture device 6 and a memory 11 of the control device 7, in particular as a function of an activation condition. The activation unit 10 has access to various other functional units, for example, including a situation capture unit 12, an interface 13 for the receipt of additional information, in particular image data from an imaging device for monitoring a minimally invasive intervention and/or position data from a position determination system for the medical instrument 2, and a user interaction unit 14.

The situation capture unit 12 mentioned above is configured to capture an intervention situation on various occasions, which, as explained in greater detail below, may be based on a variety of data or information. In particular, it is able to assign intervention situations to captured operator movement patterns.

The memory 11 is configured to store various pieces of information assigned to one another in a storage device 29 of the control device 7, in particular for storage of a captured operator movement pattern together with the assigned intervention situation, in particular as a situation dataset, and a piece of operator information describing the operator who executed the operator movement pattern. Success information describing the success of the treatment and/or the efficiency of the minimally invasive intervention in which the respective operator movement pattern was recorded may also be stored and at later points in time assigned by the memory 11 to such overall datasets including operator movement pattern (in particular thus the corresponding capture data), intervention situation (in particular thus the situation dataset) and operator (in particular thus operator information).

Such success information may be provided by an evaluation unit 15, which may evaluate image data and/or further material (received via the interface 13) for the minimally invasive intervention for determining the success information. Other sources for the at least partial determination of the success information are the user interaction unit 14 or else directly via the interface 13.

The evaluation unit 15 is further configured to evaluate, (e.g., statistically while considering the success information), operator movement patterns assigned to the same intervention situation and the same operator, in order to determine evaluation movement patterns, which are likewise stored in the storage device 29, in particular, by the memory 11.

The control device 7 further includes a trigger unit 16 that is active when the robot 8 is used and which evaluates intervention situations recorded by the situation detection unit 12 to determine whether a trigger condition is fulfilled. The trigger condition ultimately indicates that an intervention situation, in particular the start of an intervention situation, is present, in which a recorded operator movement pattern or an evaluation movement pattern may be expediently implemented by the robot 8 as a control movement pattern via control commands using the control unit 9. The selection of the control movement pattern is made using a situation unit 17, which then also correspondingly passes this to the control unit 9.

The interaction of the functional units are now explained in greater detail in respect of the method in FIG. 3. Accordingly, the use of the medical instrument arrangement 1 may be divided into two application phases 18, 19, namely a first application phase 18 (training phase) and a second application phase 19 (recall phase).

In the first application phase 18, in act S0 in a minimally invasive intervention on a subject 5, (e.g., the phantom or another subject), the activation unit 10 monitors whether the activation condition is fulfilled. Several possible preconditions exist for this. Thus, it is possible for an operator to start a movement recording manually via the user interaction unit 14, for example, if the operator is working with the phantom 5 and hence otherwise an analysis is impeded.

The minimally invasive intervention may be monitored in the activation unit 10. In one example, the activation unit 10 may check in respect of the activation condition whether a current intervention situation determined by the situation capture unit 12 is contained in a list of intervention situations for which operator movement patterns are to be recorded. It is further possible, in particular if during the entire minimally invasive intervention the operational capture device 6 supplies capture data, which may be stored in a circulating memory. It may then be provided for a current operating and/or instrument movement pattern to be compared with normal patterns that describe a normal feed operation of the instrument, for example, in that correlation values are determined for the normal patterns and the activation condition is fulfilled for all normal patterns if the value falls below a threshold value.

However, current operating and/or instrument movement patterns may alternatively or additionally also be captured differently, in particular with special sensor systems, for example a camera directed onto the operator's hands. It is also possible to evaluate image data relating to the movement of the tip of the instrument, as well as position data of the medical instrument, in order to be able to detect unusual operating and/or instrument movement patterns deviating from normal patterns.

In an alternative embodiment, it is also conceivable for the capture data to be detected continuously and subsequently to be assigned to intervention situations, and for the operator movement patterns for corresponding time intervals to be extracted.

On fulfillment of the activation condition in act S0, in act S1, the capture data is captured for the corresponding operator movement pattern. Then, in act S2, the situation capture unit 12 is used to assign an intervention situation to the operator movement pattern. In this case, the order of acts S1 and S2 may be reversed, because it may already be known in connection with the activation which intervention situation is present or how it may be described.

Whereas it is conceivable for an intervention situation to be determined on the basis of an operator input, in particular using the user interaction unit 14, this is, as already described, mainly expedient for the use of the phantom 5. The intervention situation may be captured automatically. In this connection, a situation dataset is particularly advantageously compiled which describes the position of the medical instrument 2, in particular of the tip thereof, relative to the surrounding anatomy. This may explicitly be image data, for example, image data employed in the monitoring of the minimally invasive intervention. It is thus conceivable, on fulfillment of the activation condition, for image data and position data of the medical instrument 2 to be stored in the situation dataset and to be assigned to the operator movement pattern to be recorded. However, it is also conceivable to explicitly determine against which anatomical background the medical instrument 2 is located or whether a particular landmark, which for example may also be represented in an overlay, is reached by the instrument. Such a landmark may for example be a total vascular occlusion, a bifurcation, or a vascular branch.

Different variants for the concrete determination of the current intervention situation are conceivable, e.g., side by side. Thus, for example, a trained classification function may be employed and/or a comparison may be carried out if situation datasets are used.

In act S3, the operator movement pattern, (in particular thus the corresponding capture data, the intervention situation, in particular thus the situation dataset, and operator information describing the operator, since in this case the work is operator-specific), are stored by the memory 11 in the storage device 29 as an overall dataset.

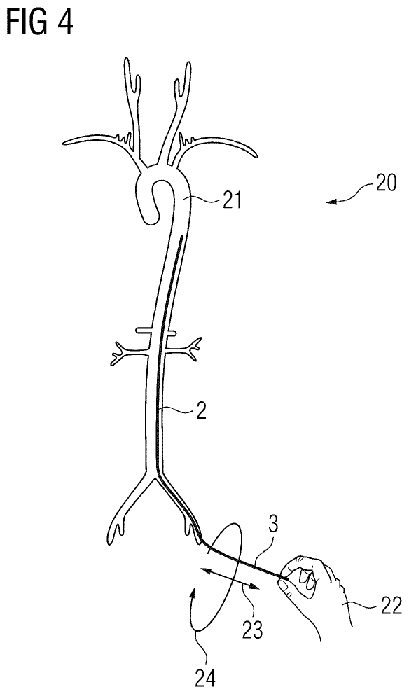

Acts S1 to S3 are illustrated and explained schematically by FIGS. 4 and 5. FIG. 4 shows the medical instrument 2 introduced into a subject 20 in an intervention situation, which in this case is navigated in a vascular system 21. The operator's hand 22 is shown in the operational section 3, which brings about movements of the operational section 3 and thus of the medical instrument 2, in this case translations (cf. arrow 23) and rotations (cf. arrow 24). FIG. 5 shows by way of example the courses 25, 26 of rotation (R(t)) and translation (T(t)) for an operator movement pattern.

Returning to FIG. 3, in act S4, on conclusion of the minimally invasive intervention, the success information may be determined by the evaluation unit 15, for example, in that image data and/or other additional information describing the status after performance of the operator movement pattern is automatically evaluated. The success information for the operator movement pattern describes its efficiency and/or success of the treatment, (e.g., continuously), for example, as a percentage/efficiency variable. For example, the success information may indicate the extent to which a CTO has been dissolved and/or how quickly it was possible to continue navigating normally and/or whether or to what extent unwanted effects, such as punctures, occurred. In act S5, the memory stores the success information in the overall dataset of the operator movement pattern.

As is indicated by the dashed arrow 27, further operator movement patterns may now be captured, in particular also for the same intervention situation and the same operator.

In act S6, the evaluation unit 15 is used to determine evaluation movement patterns for all intervention situations and operators for which multiple operator movement patterns are present. To this end, the multiple operator movement patterns are evaluated statistically, wherein the success information is correspondingly used. Thus, an optimal movement pattern for the intervention situation and the operator may be determined, which in statistical terms may produce the best success of the treatment and/or the best efficiency. The evaluation may include weighted averaging and/or the identification of efficient and/or characterizing partial movement patterns. In particular, in an update after the use of determined evaluation movement patterns these too may be used with corresponding success information in the evaluation then to be updated.

In act S7, the evaluation movement patterns with the assigned intervention situations and operators are stored in the storage device 29 using the memory 11.

In the second application phase 19, known as the “recall phase,” in act S8, during an intervention with the robot 8, the situation capture unit 12 constantly monitors, on the basis of various information, but in particular image data and position data of the medical instrument 2, which intervention situation is currently present.

This is used in act S9 by the trigger unit 16 to check in the trigger condition whether for this present intervention situation usable operator movement patterns or evaluation movement patterns are present in the storage device 29, in particular those for the current operator. If this is the case, i.e., the trigger condition is fulfilled, in act S10, the situation unit 17 selects the evaluation movement pattern suitable for the intervention situation and the operator or, if no evaluation movement pattern is present, operator movement patterns as control movement patterns.

In act S11, the control movement pattern is suggested to the operator for the performance using the user interaction unit 14, wherein, if the operator desires the performance, the control unit 9 performs the control movement pattern using control commands in act S12. There may of course also be repetitions in the application method 19, in particular also within a single minimally invasive intervention.

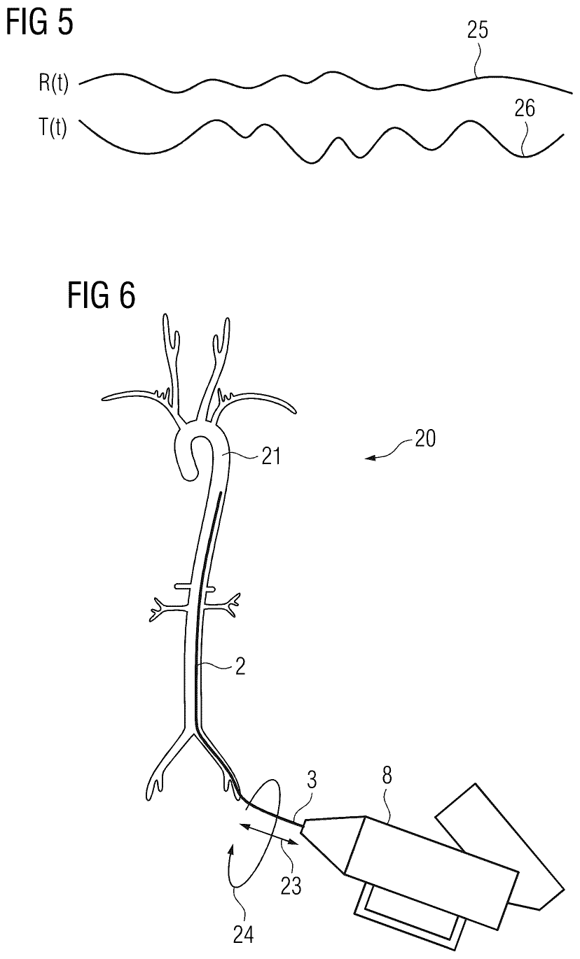

This second application phase is illustrated schematically in FIG. 6, where now instead of the hand 22 the robot 8 performs the translations (arrow 23) and the rotations (arrow 24), for example using the courses 25, 26.

It may be noted in conclusion that the control device 7, cf. FIG. 2, may optionally also have an adjustment unit 28 which adjusts operator movement patterns and/or evaluation movement patterns to restrictions in respect of the robot 8 and for the protection of the subject 20.

It is to be understood that the elements and features recited in the appended claims may be combined in different ways to produce new claims that likewise fall within the scope of the present disclosure. Thus, whereas the dependent claims appended below depend on only a single independent or dependent claim, it is to be understood that these dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A medical instrument arrangement for minimally invasive examination and/or treatment of a subject, the medical instrument arrangement comprising:
a robot for active manipulation of a medical instrument configured to be introduced into the subject, wherein the medical instrument comprises a proximal operational section for the active manipulation;
an operational capture device for capturing operator movement patterns of the proximal operational section of the medical instrument generated manually; and
a control device configured to:
determine an intervention situation to be assigned to captured operator movement patterns;
store the captured operator movement patterns with the intervention situation in a memory;
automatically select and/or generate a control movement pattern from stored operator movement patterns as a function of the intervention situation;
providing the control movement pattern as control commands for controlling the robot; and
control the robot in accordance with the control commands for generating control movements of the proximal operational section.

2. The medical instrument arrangement of claim 1, wherein the control device is configured to capture the operator movement patterns directly on the proximal operational section of the medical instrument.

3. The medical instrument arrangement of claim 1, wherein the control commands comprise a course of rotation and course of translation and/or course of curvature, to be implemented by the control device for the proximal operational section of the medical instrument.

4. The medical instrument arrangement of claim 1, wherein the control device is configured for an operator-related selection and/or the generation of the control movement pattern based on the stored operator movement patterns relating to a current operator.

5. The medical instrument arrangement of claim 1, wherein the control device is further configured to activate the storing of the captured operator movement patterns on fulfillment of an activation condition, which evaluates a current intervention situation and/or a current operating pattern and/or instrument movement pattern of the medical instrument captured by the control device.

6. The medical instrument arrangement of claim 5, wherein the control device is configured, for the evaluation of the activation condition, to compare the current operating pattern and/or the instrument movement pattern with at least one predefined normal pattern, and
wherein, in a case of a deviation for each normal pattern exceeding a threshold value, the activation condition is fulfilled.

7. The medical instrument arrangement of claim 6, wherein the at least one predefined normal pattern describes a normal feed operation of the instrument.

8. The medical instrument arrangement of claim 1, wherein the control device is further configured to:
capture the intervention situation for an operator movement pattern for a definition of a situation dataset that describes at least a position of the medical instrument relative to a surrounding anatomy; and
determine a present intervention situation by comparison with stored situation datasets.

9. The medical instrument arrangement of claim 8, wherein the surrounding anatomy comprises at least one landmark.

10. The medical instrument arrangement of claim 1, wherein the control device is further configured for use of at least one classification function for the determining of the intervention situation.

11. The medical instrument arrangement of claim 10, wherein the at least one classification function is a trained classification function.

12. The medical instrument arrangement of claim 1, wherein the control device, on fulfillment of a trigger condition evaluating a present intervention situation, is configured to trigger the selection and/or the generation of the control movement pattern.

13. The medical instrument arrangement of claim 1, wherein the control device is configured to statistically evaluate multiple operator movement patterns present in the memory for an identical intervention situation for a determination of a selectable evaluation movement pattern for the intervention situation.

14. The medical instrument arrangement of claim 13, wherein the control device is further configured to store success information describing a success of the respective captured operator movement pattern, as a continuous efficiency variable, for each captured operator movement pattern, and
wherein the control device is configured to determine the evaluation movement pattern as an optimal movement pattern using the success information.

15. The medical instrument arrangement of claim 14, wherein the success information is time-offset success information.

16. The medical instrument arrangement of claim 14, wherein the control device is further configured to evaluate image data of the medical instrument and a surrounding anatomy recorded during and/or after a performance of the operator movement pattern for an at least partially automatic determination of the success information.

17. The medical instrument arrangement of claim 1, wherein the control device is further configured to adjust the operator movement patterns in respect of at least one restriction of movement relating to the robot and/or a protection of the subject.

18. A method for operating a medical instrument arrangement for a minimally invasive examination and/or treatment of a subject, the medical instrument arrangement having a medical instrument introduced into the subject, wherein a proximal operational section of the medical instrument is configured to move the medical instrument, the method comprising:
capturing, by the medical instrument arrangement, operator movement patterns of the proximal operational section of the medical instrument generated manually, directly on the proximal operational section;
assigning, by the medical instrument arrangement, an intervention situation to each captured operator movement pattern;
storing, by the medical instrument arrangement, the captured operator movement patterns with the intervention situation; and
automatically selecting and/or generating, by the medical instrument arrangement, in a further intervention with the medical instrument and a robot for an active manipulation of the medical instrument on the proximal operational section, a control movement pattern from the captured operator movement patterns as a function of a determined intervention situation; and
providing, by the medical instrument arrangement, the control movement pattern as control commands for the robot.

19. A computer program or a non-transitory electronically readable data medium storing the computer program, which, when the computer program is executed on a control device of a medical instrument arrangement, causes the control device to:
capture operator movement patterns of a proximal operational section of a medical instrument of the medical instrument arrangement generated manually, directly on the proximal operational section;
assign an intervention situation to each captured operator movement pattern;
store the captured operator movement patterns with the intervention situation; and
automatically select and/or generate, in a further intervention with the medical instrument and a robot for an active manipulation of the medical instrument on the proximal operational section, a control movement pattern from the captured operator movement patterns as a function of a determined intervention situation; and
provide the control movement pattern as control commands for the robot.
